(19) United States
(12) Patent Application Publication
(10) Pub. No.: US 2024/0082360 A1
(43) Pub. Date: Mar. 14, 2024

(54) METHODS TO TREAT HEPATITIS DELTA VIRAL INFECTIONS

(71) Applicant: Eiger Biopharmaceuticals, Inc., Palo Alto, CA (US)

(72) Inventors: Ingrid CHOONG, Palo Alto, CA (US); Jeffrey S. GLENN, Palo Alto, CA (US)

(21) Appl. No.: 17/754,587

(22) PCT Filed: Oct. 15, 2020
(86) PCT No.: PCT/US2020/055714

§ 371 Date: Apr. 6, 2022

Related U.S. Application Data
(60) Provisional application No. 63/070,047, filed on Aug. 25, 2020.
(60) Provisional application No. 63/014,774, filed on Apr. 24, 2020.
(60) Provisional application No. 62/915,933, filed on Oct. 16, 2019.

(51) Int. Cl. A61K 38/21; A61K 31/427; A61K 31/4545; A61K 31/7052; A61P 31/14

(52) U.S. Cl. CPC A61K 38/212; A61K 31/427; A61K 31/4545; A61K 31/7052; A61P 31/14

(57) ABSTRACT

Methods of treating a hepatitis delta virus (HDV) infection in a human subject are provided. In some embodiments, the method comprises subcutaneously administering to the subject a therapeutically effective amount of pegylated interferon lambda-1a in combination with lonafarnib and ritonavir for at least 24 weeks. In another aspect, provided herein are methods of treating HDV infections in a subject, comprising the following drug regimen subcutaneously administering to the subject a therapeutically effective amount of pegylated interferon lambda-1a once per week, and administering lonafarnib and ritonavir daily until one or more of a sustained rHDV viral load is reached, or a decrease in HDV RNA to undetectable levels, or for 12 weeks, or for 24 weeks, or for 48 weeks.

### RELATED APPLICATIONS

This application claims priority to U.S. Provisional Application 63/014,774, entitled “Methods to Treat Hepatitis Delta Viral Infections,” filed Apr. 24, 2020, to U.S. Provisional Application 62/915,933, entitled “Methods to Treat Hepatitis Delta Viral Infections,” filed Oct. 16, 2019, and to U.S. Provisional Application 63/070,047, entitled “Methods to Treat Hepatitis Delta Viral Infections,” filed Aug. 25, 2020.

### FIELD

The present invention provides methods for treating viral hepatitis resulting from hepatitis delta virus infection, and so relates to the fields of chemistry, medicinal chemistry, medicine, molecular biology, and pharmacology.

### BACKGROUND

Hepatitis delta virus (HDV) infection is the most aggressive form of human chronic viral hepatitis. Chronic HDV and HBV co-infection worsens preexisting HBV-related liver damage and leads to liver cirrhosis, hepatic decompensation, and hepatocellular carcinoma. See, Negro, Cold Spring Harb. Perspect. Med., 2014, 4:a021550; Honer zu Siederdissen, Visc. Med., 2016, 32:86-94; Lau, Hepatology, 1999, 30:546-549. Subjects co-infected with both HDV and HBV are more likely to die of complications from liver disease compared to subjects infected with HBV alone. See, Alavian et al., J. Res. Med. Sci., 2012, 17:967-974.

Interferon alpha therapy for the treatment of HDV has been described. In Hep-Net International Delta Intervention Trial 1 and 2 (HIDIT-1 and -2) studies, published in 2011 and 2019, respectively, it was found that 23-31% of subjects receiving peginterferon alfa-2a therapy achieved undetectable HDV RNA 24 weeks after the end of treatment, measured using a quantitative HDV RNA assay with a lower limit of detection (LOD) of HDV RNA of 120 copies/mL of serum (Mederacke et al, 2010). A conversion factor was later determined to be one copy per mL in this assay, equal to 62 IU per mL (Wedemeyer et al, 2019a). Applying the conversion factor results in limit of detection of 930 IU/mL. Thus, the actual undetectable HDV RNA rate is likely much lower than 23-31% given the high limit of detection of this laboratory assay. Wedemeyer et al., N Engl J Med, 2011, 364:322-331 and Wedemeyer 2019 ref.

The unsatisfactory anti-HDV effects of interferon alfa was reported in another clinical study (MYR203 Study) at EASL 2019 demonstrating that 48 weeks of treatment with interferon alfa 180 mcg monotherapy as an active control group resulted in only 13% of the HDV patients with undetectable HDV RNA at the end of 48 weeks treatment (Wedemeyer et al, 2019b) using the Robogene® 2.0 HDV RNA quantitative assay. This undetectability was not maintained at 24 weeks post-treatment follow-up where 0% of patients had undetectable HDV RNA.

In contrast to interferon alpha, which mediates its effects by signaling through interferon alpha receptors that are widely expressed by many different cell types, interferon lambda signals through a different class of receptors (the “interferon lambda receptors”) that have a restricted cellular expression pattern. Interferon lambda also exhibits distinct antiviral activities from interferon alpha, due in part to the differences in expression of the interferon receptors. In a comparative study of pegylated interferon alfa and a pegylated interferon lambda for the treatment of HBV (Chan et al., J. Hepatology, 2016, 64:1011-1019), it was found that although pegylated interferon lambda produced more pronounced declines in viremia as compared to pegylated interferon alfa at the midpoint of treatment (24 weeks), by the end of the treatment period there was no difference between pegylated interferon alfa and pegylated interferon lambda treatment, and post-treatment there was a greater virologic rebound in the pegylated interferon lambda treatment group. HBV/HDV co-infected mice receiving pegylated interferon alfa for four weeks exhibited a 2.2 log reduction in HDV-RNA levels, while mice receiving pegylated interferon lambda for four weeks exhibited a 1.5 log reduction in HDV-RNA levels (Giersch et al., 2013).

To date, the efficacy of long-term pegylated interferon lambda therapy in combination with lonafarnib and ritonavir for the treatment of HDV has not been described. There continues to be an ongoing need for agents to treat HDV infection.

There is still a need in the art for a safe and effective treatment for HDV.

### SUMMARY

In one aspect, methods of treating a hepatitis delta virus (HDV) infection in a human subject are provided.

In another aspect, provided herein are methods of treating HDV infections in a subject, comprising the following drug regimen subcutaneously administering to the subject a therapeutically effective amount of pegylated interferon lambda-1a once per week, and administering lonafarnib and ritonavir daily until one or more of a sustained reduction of HDV viral load is reached, or a decrease in HDV RNA to undetectable levels, or for 12 weeks, or for 24 weeks, or for 48 weeks.

In another aspect, provided herein are methods of treating a hepatitis delta virus (HDV) in a subject comprising the following drug regimen administering to the subject a therapeutically effective amount of each of interferon lambda, lonafarnib, and ritonavir for a first treatment period, wherein the interferon lambda is administered at a first interferon lambda dosage, the lonafarnib is administered at a first lonafarnib dosage, and the ritonavir is administered at a first ritonavir dosage; and administering to the subject a therapeutically effective amount of each of lonafarnib and ritonavir for a second treatment period after the first treatment period, wherein the lonafarnib is administered at a second lonafarnib dosage and the ritonavir is administered at a second ritonavir dosage.

In one embodiment, the drug regimen is administered for at least 12 weeks, or 24 weeks, or 36 weeks, or 48 weeks, or 54 weeks, or between 12 weeks and 48 weeks.

In one embodiment, the pegylated interferon lambda-1a is administered at a dose of 180 micrograms once a week (QW) or 90 micrograms twice per week; or 80 micrograms twice per week, or 180 micrograms per week.

In one embodiment, the pegylated interferon lambda-1a is administered at a dose of 120 micrograms QW, or 60 micrograms twice per week, or 70 micrograms twice per week, or 120 micrograms per week.

In one embodiment, there is a decrease of HDV RNA by >2 log₁₀ from baseline at 24 weeks after initiation of administration.

In one embodiment, a subject has undetectable HDV RNA in serum at 12 weeks after initiating administration and/or at 24 week post-treatment follow-up, e.g., 24 weeks after a last administration.

In one embodiment, a subject has a reduction in histologic inflammatory scores (modified HAI) by at least two points at end of treatment with no progression in histologic fibrosis. The reduction may be as compared to a baseline.

In one embodiment, a subject has normalization of serum ALT at one or more of the end of therapy, at week 12 of post-therapy follow-up and at week 24 of post-therapy follow-up. In one embodiment, ALT levels are reduced in females to <20 IU/L and <31 IU/L in males.

In one embodiment, a subject has reduction in serum ALT by >50% of baseline at one or more of week 12 of post therapy follow up and week 24 of post-therapy follow up.

In one embodiment, a subject has reduction in hepatic venous pressure gradient (HVPG) measurements by >25% of baseline or normalization of HVPG (<5 mm Hg) at end of therapy.

In one embodiment, a subject has reduction in Fibroscan® transient elastography values by >25% of baseline at end of therapy.

In one embodiment, a subject has loss of HBsAg from the serum at one or more of the end of therapy, at week 12 of post-therapy follow-up or at week 24 of post-therapy follow-up.

In one embodiment, a subject has changes in quantitative HBsAg levels at one or more of the end of therapy and at week 24 of post-therapy follow up, compared to baseline.

In one embodiment, after 12 weeks of therapy at least about 81% of subjects had a median HDV RNA decline from baseline of at least 3.6 log IU/mL.

In one embodiment, after 12 weeks of therapy a majority of subjects will have a median RNA decline of between about 2.6-4.2 log IU/mL.

In one embodiment, after 12 weeks of therapy at least about 24% of subjects have undetectable HDV RNA.

In one embodiment, after 12 weeks of therapy at least about 24% of subjects have HDV RNA below the lower limit of quantification (BLOQ).

In one embodiment, at the end of therapy at least about 73% of subjects have HDV RNA decline of at least about 3.4 log IU/mL.

In one embodiment, at the end of therapy a majority of subjects have HDV RNA decline of from about 2.9-about 4.5 log IU/mL.

In one embodiment, at the end of therapy at least 37% of subjects achieve undetectable HDV RNA

In one embodiment, at the end of therapy at least about 16% of subjects achieve BLOQ.

In one embodiment, at the end of therapy at least about 95% of subjects achieved >2 log decline of HDV RNA during 24 weeks of therapy.

In one embodiment, greater than 50% of subjects achieve undetectable or BLOQ HDV RNA levels.

In one embodiment, the method further comprises administering to the subject a nucleoside analog or nucleotide analog. In one embodiment, the nucleoside analog or nucleotide analog is lamuvidine, adefovir, telbivudine, entecavir, or tenofovir. In one embodiment, the subject has compensated liver disease with or without cirrhosis. In one embodiment, the subject has compensated liver disease with cirrhosis.

In another aspect, provided herein are methods of assessing impact of a therapeutic agent targeting hepatitis beta virus (HBV) on an hepatitis delta virus (HDV) infection in a subject having a co-infection of HBV and HDV. The methods include the steps of administering the therapeutic agent to the subject for a treatment period measuring an amount of HBsAg in a biological sample from the subject after the treatment period; and determining if the amount of HBsAg in the biological sample is undetectable; wherein if the amount of HBsAg is undetectable, the therapeutic agent impacts the HDV infection.

In one embodiment, the method also includes determining a baseline amount HDV RNA in the subject; measuring an amount of HDV RNA in a biological sample from the subject after the treatment period; and determining if the amount of HDV RNA in the biological sample is reduced relative to the baseline amount of HDV RNA.

### DETAILED DESCRIPTION

### Definitions

The terminology used herein is for the purpose of describing particular embodiments only, and is not intended to be limiting, because the scope of the present invention will be limited only by the appended claims. Unless defined otherwise, all technical and scientific terms used herein have the same meaning as commonly understood by one of ordinary skill in the art to which this invention belongs. In this specification and in the claims that follow, reference will be made to a number of terms that shall be defined to have the following meanings unless a contrary intention is apparent. In some cases, terms with commonly understood meanings are defined herein for clarity and/or for ready reference, and the inclusion of such definitions herein should not be construed as representing a substantial difference over the definition of the term as generally understood in the art.

Although any methods and materials similar or equivalent to those described herein can be used in the practice or testing of the present invention, the preferred methods, devices, and materials are now described. All technical and patent publications cited herein are incorporated herein by reference in their entirety. Nothing herein is to be construed as an admission that the invention is not entitled to antedate such disclosure by virtue of prior invention.

All numerical designations, e.g., pH, temperature, time, concentration, and molecular weight, including ranges, are approximations which are varied by increments of plus or minus 0.1 or 1.0, as appropriate. It is to be understood, although not always explicitly stated that all numerical designations are preceded by the term “about.”

The singular forms “a,” “an,” and “the” include plural referents unless the context clearly dictates otherwise. Thus, for example, reference to “a compound” includes a plurality of compounds.

The term “administration” refers to introducing a compound, a composition, or an agent of the present disclosure into a host, such as a human. In the context of the present disclosure, one preferred route of administration of the agents is subcutaneous administration. Other routes of administration include intravenous administration and oral administration.

The term “baseline,” unless otherwise specified or apparent from context, refers to a measurement (of, e.g., viral load, subject condition, ALT level) made prior to a course of therapy.

The term “comprising” is intended to mean that the compounds, compositions and methods include the recited elements, but does not exclude others. “Consisting essentially of” when used to define compounds, compositions and methods, shall mean excluding other elements that would materially affect the basic and novel characteristics of the claimed invention. Embodiments defined by each of these transition terms are within the scope of this invention.

The terms “course of treatment” and “course of therapy” are used interchangeably herein, and refer to the medical interventions made after a subject is diagnosed, e.g., as being infected with HDV and in need of medical intervention. Medical interventions include, without limitation, the administration of drugs for a period of time, typically, for HDV infected subjects, at least one and typically several or many months or even years.

The term “HDV RNA viral load” or “viral load” of a human serum or plasma sample refers to the amount of HDV RNA in a given amount of a human serum or plasma sample. HDV RNA is generally detected by quantitative real-time reverse transcription-polymerase chain reaction (qRT-PCR) assays. In such assays, the amount of signal generated during the assay is proportional to the amount of HDV RNA in the sample. The signal from the test sample is compared to that of a dilution series of a quantified Hepatitis Delta RNA standard, and a copy number of genome copies is calculated. See, e.g., Kodani et al., 2013, J. Virol. Methods, 193(2), 531; Karatayli et al., 2014, J. Clin. Virol, 60(1), 11. HDV RNA viral load may be reported as RNA copies per mL serum (or plasma) or using International Units (IU) per mL serum (or plasma). See, Chudy et al., 2013, Collaborative Study to establish a World Health Organization International standard for hepatitis D virus RNA for nucleic acid amplification technique (NAT)-based assays.” WHO Expert Committee on Biological Standardization WHO/BS/2013.2227. A commercially available assay is available from ARUP Laboratories (Salt Lake City, UT). The limit of detection for the ARUP HDV RNA assay has been reported to be 31 IU/mL. Analytik Jena AG (Germany) offers the RoboGene® HDV RNA Quantification Kit 2.0, which is CE-IVD certified with WHO standard references to assess the response to antiviral treatment. The limit of detection for the RoboGene® assay is reported to be 6 IU/mL. Reference to a “viral load” without specified units (e.g., “a viral load of less than 100”) refers to copies of HDV RNA per mL serum, unless otherwise indicated or apparent from context. Unless otherwise specified, reference to “below the level of detection” means below 8 IU/mL.

HDV levels are generally presented using log₁₀ units. HDV RNA levels may be presented in units of “RNA copies per mL” or as “International Units (IU) per mL.” See, Chudy et al., 2013, Collaborative Study to establish a World Health Organization International standard for hepatitis D virus RNA for nucleic acid amplification technique (NAT)-based assays.” WHO Expert Committee on Biological Standardization WHO/BS/2013.2227. Both units are used in this specification. As used herein, recitation of “HDV RNA copies per mL,” (when not otherwise specified and not including discussions related to clinical trial results, e.g., as presented in the examples) should be read, for purposes of written description or basis, as referring to “HDV RNA copies/mL or HDV IU/mL.” Where a specific quantity of HDV RNA copies per mL is recited, a multiplier of 1.2 may be applied, for the purposes of written description and support, to convert the quantity of HDV RNA copies/mL to the quantity of IU/mL. For example, “120 HDV RNA copies per mL” should be read as “120 copies/mL or 100 IU/mL.” Changes in HDV RNA levels may be represented as a “log reduction” following the normal conventions of virology.

The term “HDV infection” with respect to a human (host) refers to the fact that the host is suffering from HDV infection. Typically, an HDV infected human host will have a viral load of HDV RNA of at least about 2 log₁₀ HDV RNA copies/mL of host serum or plasma or 10² copies of HDV-RNA/mL of host serum or plasma, often at least about 3 log₁₀ HDV RNA copies/mL of host serum or plasma or 10³ copies of HDV-RNA/mL of host serum or plasma, and, often, especially for subjects not on any therapy, at least about 4 log₁₀ HDV RNA copies/mL of host serum or plasma or 10⁴ copies of HDV-RNA/mL of host serum or plasma, such as about 4 log₁₀ HDV RNA copies/mL of host serum or plasma to 8 log₁₀ HDV RNA copies/mL of host serum or plasma or 10⁴ to 10⁸ copies of HDV-RNA/mL of host serum or plasma. As used herein, the term “chronic HDV infection” with respect to a human host refers to an HDV infection that has persisted in the human host for at least 6 months, as documented by a positive HDV antibody (Ab) test and/or detectable HDV RNA by qRT-PCR. Diagnosis and pathogenesis of HDV is described, for example, in Wedemeyer et al., Nat. Rev. Gastroenterol. Hepatol, 2010, 7:31-40.

The term “lower limit of quantification” refers to the lowest concentration of a substance of analyte (e.g., a viral titer) that can be reliably quantified by a particular assay within a stated confidence limit.

The terms “subject,” “host,” or “patient,” are used interchangeably and refer to a human infected with HDV, including subjects previously infected with HDV in whom virus has cleared.

The term “pharmaceutical composition” is meant to encompass a composition suitable for administration to a subject. In general, a “pharmaceutical composition” is sterile, and preferably free of contaminants that are capable of eliciting an undesirable response within the subject (e.g., the compound(s) in the pharmaceutical composition is pharmaceutical grade). Pharmaceutical compositions can be designed for administration to subjects or subjects in need thereof via a number of different routes of administration including oral, intravenous, buccal, rectal, parenteral, intraperitoneal, intradermal, intratracheal, intramuscular, subcutaneous, inhalational, and the like.

A “sustained reduction” of HDV viral load means a reduction of viral load (e.g., a decrease of at least 1.5 log₁₀ HDV RNA IU/mL serum, at least 2.0 log₁₀ HDV RNA copies/mL serum, or at least 2.5 log₁₀ HDV RNA IU/mL serum, or a decrease in HDV RNA to undetectable levels) for a period time (e.g., 1 month, 3 months, 6 months, 1 year or longer). The sustained reduction may be a period of time during which the course of treatment is still ongoing or a period of time after the course of treatment is finished.

The term “therapeutically effective amount” as used herein refers to that amount of an embodiment of the agent (e.g., a compound, inhibitory agent, or drug) being administered that will treat to some extent a disease, disorder, or condition, e.g., relieve one or more of the symptoms of the disease, i.e., infection, being treated, and/or that amount that will prevent, to some extent, one or more of the symptoms of the disease, i.e., infection, that the subject being treated has or is at risk of developing.

The terms “treatment,” “treating,” and “treat” are defined as acting upon a disease, disorder, or condition with an agent to reduce or ameliorate the pharmacologic and/or physiologic effects of the disease, disorder, or condition and/or its symptoms. “Treatment,” as used herein, covers any treatment of a disease in a human subject, and includes: (a) reducing the risk of occurrence of the disease in a subject determined to be predisposed to the disease but not yet diagnosed as infected with the disease, (b) impeding the development of the disease, and/or (c) relieving the disease, i.e., causing regression of the disease and/or relieving one or more disease symptoms. “Treatment” is also meant to encompass delivery of an inhibiting agent to provide a pharmacologic effect, even in the absence of a disease or condition. For example, “treatment” encompasses delivery of an agent that provides for enhanced or desirable effects in the subject (e.g., reduction of viral load, reduction of disease symptoms, etc.).

The terms “undetectable” or “below the level of detection” or “BLD”, as used with reference to HDV RNA levels, means that no HDV RNA copies can be detected by the assay methodology employed. In some embodiments, the assay is quantitative RT-PCR.

In one aspect, the present disclosure provides methods of treating HDV infection by administering interferon lambda therapy to an HDV-infected subject. In some embodiments, a pegylated form of interferon lambda (e.g., pegylated interferon lambda-1a (“LMD”)) is administered. In some embodiments, subjects receiving interferon lambda therapy (e.g., pegylated interferon lambda therapy) are also treated with an antiviral nucleoside or nucleotide analog (e.g., an anti-HBV nucleotide or nucleoside analog). In some embodiments, subjects receiving interferon lambda therapy (e.g., pegylated interferon lambda therapy) are also treated with lonafarnib (“LNF”) therapy or lonafarnib and ritonavir (“RTV”) therapy, e.g., for the duration of the interferon lambda therapy or during a portion of the time that interferon lambda therapy is administered. In some embodiments, subjects receiving interferon lambda therapy (e.g., pegylated interferon lambda therapy) are not administered an antiviral nucleoside or nucleotide analog therapy. In some embodiments, subjects receiving interferon lambda therapy (e.g., pegylated interferon lambda therapy) are not administered lonafarnib therapy or lonafarnib and ritonavir therapy.

### Interferon Lambda, Lonafarnib and Ritonavir

Interferons are polypeptides that inhibit viral replication and cellular proliferation and modulate immune response. Based on the type of receptor through which they signal, human interferons have been classified into three major types (Types I, II, and III). All type I IFNs bind to a specific cell surface receptor complex known as the IFN-alpha receptor (IFNAR) that consists of IFNAR1 and IFNAR2 chains. The type I interferons present in humans are IFN-alpha, IFN-beta, IFN-epsilon, and IFN-omega. Type II IFNs bind to IFN-gamma receptor (IFNGR) that consists of IFNGR1 and IFNGR2 chains. The type II interferon in humans is IFN-gamma. The type III interferon group consists of three IFN-lambda molecules called IFN-lambdal, IFN-lambda2 and IFN-lambda3 (also called IL29, IL28A, and IL28B, respectively). These IFNs signal through a receptor complex consisting of IL10R2 (also called CRF2-4) and IFNLR1 (also called CRF2-12).

The term “interferon-lambda” or “IFN-λ” as used herein includes naturally occurring IFN-λ; synthetic IFN-λ; derivatized IFN-λ (e.g., PEGylated glycosylated IFN-λ, and the like); and analogs of naturally occurring or synthetic IFN-2 In some embodiments, an IFN-λ is a derivative of IFN-λ that is derivatized (e.g., chemically modified relative to the naturally occurring peptide) to alter certain properties such as serum half-life. As such, the term “IFN-λ” includes IFN-λ derivatized with polyethylene glycol (“PEGylated IFN-λ”), and the like. PEGylated IFN-λ (e.g., PEGylated IFNλ-1a), and methods for making same, is discussed in, e.g., U.S. Pat. Nos. 6,927,040, 7,038,032, 7,135,170, 7,157,559, and 8,980,245 and PCT Publication Nos. WO 2005/097165, WO 2007/012033, WO 2007/013944 and WO 2007/041713; all of which are herein incorporated by reference in their entirety. In some embodiments, the IFN-λ is an IFN-λ as disclosed in PCT/US2017/018466, which is incorporated by reference herein in its entirety. In some embodiments, the pegylated IFN-λ-1a has the structure described in U.S. Pat. No. 7,157,559, which is incorporated by reference herein in its entirety.

In some embodiments, an interferon for use in a therapeutic method as described herein is a pegylated IFN-21 (e.g., pegylated IFNλ-1a), pegylated IFNλ-2, or pegylated IFNλ-3. In some embodiments, the interferon is pegylated IFNλ1 (e.g., pegylated 1a).

Lonafarnib therapy for the treatment of HDV is disclosed in US 2017/0042862, incorporated by reference herein.

In some embodiments, the lonafarnib component of the therapy is administered at a total daily dose of 50-200 mg per day, e.g., at least 50 mg per day, at least 75 mg per day, at least 100 mg per day, at least 150 mg per day, or at least 200 mg per day. Lonafarnib therapy may be administered once daily (QD) or twice daily (BID). In some embodiments, lonafarnib is administered at a dose of 25 mg BID, 50 mg BID, 75 mg BID, 100 mg BID, 50 mg QD, 75 mg QD, or 100 mg QD. In some embodiments, lonafarnib therapy is initiated at the start of interferon lambda therapy or during the course of interferon lambda therapy.

A CYP3A inhibitor, such as ritonavir or cobicistat is also co-administered. In some embodiments, the CYP3A inhibitor is ritonavir. Lonafarnib and ritonavir co-therapy is disclosed in WO 2015/168648 and in WO 2017/079009, incorporated by reference herein.

In some embodiments, the lonafarnib-ritonavir portion of the therapy is at a total daily dose of 50-200 mg of lonafarnib per day (e.g., at least 50 mg per day, at least 75 mg per day, at least 100 mg per day, at least 150 mg per day, or at least 200 mg per day of lonafarnib) and 100-200 mg of ritonavir per day (e.g., at least 100 mg per day, at least 150 mg per day, or at least 200 mg per day of ritonavir). The lonafarnib and ritonavir portion of the therapy may be administered once daily (QD) or twice daily (BID). In some embodiments, the lonafarnib is at a dose of 25 mg BID, 50 mg BID, 75 mg BID, 100 mg BID, 50 mg QD, 75 mg QD, or 100 mg QD, and the ritonavir at a dose of 50 mg BID or 100 mg BID.

In some embodiments, a subject to be treated with interferon lambda, lonafarnib and ritonavir therapy as described herein is a subject having an HDV infection, e.g., an acute HDV infection or a chronic HDV infection. In some embodiments, the subject to be treated has a chronic HDV infection of at least 6 months in duration as documented by a positive HDV antibody (Ab) test, and/or detectable HDV RNA by qRT-PCR. In some embodiments, a subject to be treated with a therapeutic method described herein is a subject having an acute HDV infection, e.g., a newly diagnosed HDV infection or a HDV infection otherwise believed not to have existed in the subject for more than six months. Diagnosis and pathogenesis of HDV is described, for example, in Wedemeyer et al., Nat. Rev. Gastroenterol. Hepatol, 2010, 7:31-40. HDV is known to exist in a variety of subtypes; the methods described herein are suitable for treating all HDV subjects, regardless of HDV subtype. In some embodiments, the subject is an adult (18 years or older), and in other embodiments, the subject is pediatric.

In some embodiments, the HDV viral load of the subject is >2 log₁₀ above the lower limit of quantification (LLOQ) of the HDV RNA assay. In some embodiments, the viral load is measured at three pre-treatment points with a mean viral load of >2 log₁₀ above the LLOQ of the HDV RNA assay.

In some embodiments, a subject to be treated has a baseline viral load of at least 10² HDV RNA copies per mL serum or plasma or at least 10² HDV RNA IU/mL serum or plasma, e.g., at least 10³ HDV RNA copies per mL or at least 10³ HDV RNA IU/mL serum or plasma, at least 10⁴ HDV RNA copies per mL or at least 10⁴ HDV RNA IU/mL serum or plasma, at least 10⁵ HDV RNA copies per mL or at least 10⁵ HDV RNA IU/mL serum or plasma, at least 10⁶ HDV RNA copies per mL or at least 10⁶ HDV RNA IU/mL serum or plasma, at least 10⁷ HDV RNA copies per mL or at least 10⁷ HDV RNA IU/mL serum or plasma, or at least 10⁸ HDV RNA copies per mL or at least 10⁸ HDV RNA IU/mL serum or plasma. In some embodiments, HDV viral load is measured using serum samples from the subject. In some embodiments, HDV viral load is measured using plasma samples from the subject. In some embodiments, viral load is measured by quantitative RT-PCR. qRT-PCR assays for quantification of HDV RNA in serum or plasma are known in the art, e.g., as described above. In some embodiments, a subject to be treated has a baseline viral load that is up to about 10⁴ HDV RNA copies per mL serum or plasma or up to about 10⁴ HDV RNA IU/mL serum or plasma. In some embodiments, a subject to be treated has a baseline viral load that is up to about 10⁵ HDV RNA copies per mL serum or plasma and/or up to about 10⁵ HDV RNA IU/mL serum or plasma. In some embodiments, a subject to be treated has a baseline viral load that is up to about 10⁶ HDV RNA copies per mL serum or plasma and/or up to about 10⁶ HDV RNA IU/mL serum or plasma.

In some embodiments, HDV viral load is measured using serum samples from the subject. In some embodiments, HDV viral load is measured using plasma samples from the subject. In some embodiments, viral load is measured by quantitative RT-PCR. qRT-PCR assays for quantification of HDV RNA in serum or plasma are known in the art, e.g., as described above.

In some embodiments, a subject to be treated has one or more of: the presence of anti-HDV in serum; presence of quantifiable HDV RNA in serum at three time pre-treatment points with a mean HDV RNA level greater than 2 log₁₀ above the LLOQ of the HDV RNA assay; the demonstration of chronicity as evidenced by the presence of HDV RNA in serum for at least 6 months; or presence of anti-HDV antibody for at least 6 months.

In some embodiments, a subject to be treated exhibits one or more symptoms of liver dysfunction. In some embodiments, the subject exhibits one or more liver function parameters that are outside the normal parameters for a healthy control (e.g., a subject that is not infected with HDV and/or HBV). In some embodiments, the liver function parameter is selected from the group consisting of serum albumin, bilirubin, alanine aminotransferase (ALT), aspartate aminotransferase (AST), and prothrombin activity. In some embodiments, the subject has a serum ALT level that is at least two-fold higher than the upper limit of normal (ULN) (e.g., at least 2-fold, at least 3-fold, at least 4-fold, at least 5-fold, at least 6-fold, at least 7-fold, at least 8-fold, at least 10-fold or higher than the ULN). Liver function parameters are described in the art. See, e.g., Limdi et al., Postgrad Med J, 2003, 79:307-312. Methods of measuring these liver function parameters are known in the art.

In some embodiments, the subject has compensated liver disease (e.g., as classified according to the Child-Turcotte-Pugh Classification System) with or without liver cirrhosis. It will be recognized by a person of ordinary skill in the art that the Child-Turcotte-Pugh Classification System is used to classify the severity of liver disease and is determined by assessing serum albumin levels, bilirubin levels, international normalized ratio of prothrombin time levels, ascites formation, and encephalopathy. In some embodiments, the subject has a Child-Turcotte-Pugh score of 5-6 (class A). In some embodiments, the subject has a Child-Turcotte-Pugh score of 1-6. In some embodiments, the subject has a Child-Turcotte-Pugh score of a sub-range of 1-6, e.g., 1-2, 1-3, 2-4, 3-4, 2-5, 3-5, or 2-6. In some embodiments, the subject has compensated liver disease with liver cirrhosis. In some embodiments, the subject has compensated liver disease without liver cirrhosis.

In some embodiments, the subject is diagnosed with chronic hepatitis as determined by, for example, one or more of: a liver biopsy, a liver function test, an ultrasound, a hepatic venous pressure gradient (HVPG) measurement, an ALT level, one or more other blood tests, or an albumin level. In some embodiments, the biopsy is conducted within the 6 months before the initiation of treatment. In some embodiments, the biopsy is conducted within the 18 months before the initiation of treatment, according to the methods provided herein. In some embodiments, the biopsy is conducted within the 1 day to 24 months before the initiation of treatment. In some embodiments, the subject has evidence of chronic hepatitis based on a liver biopsy within 6 months before screening. In some embodiments, the subject has a serum alanine aminotransferase (ALT) level that is above the upper limit of normal (ULN) within 24 weeks prior to treatment and/or at the initiation of treatment, within 24 months prior to the initiation of treatment, from 24 months to 1 month prior to the initiation of treatment, or from 12 months to 1 day prior to the initiation of treatment. In various embodiments, the subject meets one or more independently selected criteria in Example 1.

### Dosing Regimens

In some embodiments, interferon lambda, lonafarnib, and ritonavir therapy comprises administering to the subject interferon lambda (e.g., pegylated interferon lambda-1a) at a dose of 180 micrograms (mcg) per week, lonafarnib at a dose of 50 mg twice daily (BID), and ritonavir at a dose of 100 mg BID. In one embodiment, the subject is administered interferon lambda at about 200 mcg to about 100 mcg QD, lonafarnib at from about 25 mg to about 50 mg BID, and ritonavir from about 50 mg to about 150 mg BID.

In some embodiments, interferon lambda, lonafarnib, and ritonavir therapy comprises administering to the subject interferon lambda at a dose of 180-120 mcg per week, lonafarnib at a dose of 50 mg BID, and ritonavir at a dose of 200 mg QD.

In some embodiments, the lambda is administered at 120 mcg per week, 110 mcg per week, 100 mcg per week, 90 mcg per week, 80 mcg per week, 120-70 mcg per week, 200-120 mcg per week, or 170-130 mcg per week. In some embodiments, interferon lambda is administered at a dose of 180 mcg QW. In some embodiments, interferon lambda is administered at a dose of 90 mcg two times per week. In some embodiments, interferon lambda is administered at a dose of 90 mcg every 3-4 days. In some embodiments, interferon lambda is administered at a dose of 80 mcg two times per week. In some embodiments, interferon lambda is administered at a dose of 80 mcg every 3-4 days. In some embodiments, interferon lambda is administered at a dose of 100-70 mcg two times per week. In some embodiments, interferon lambda is administered at a dose of 100-70 mcg every 3-4 days. In some embodiments, interferon lambda is administered at a dose of 120 mcg QW. In some embodiments, interferon lambda is administered at a dose of 80 mcg QW.

In some embodiments, a subject being treated for HDV infection receives an adjustment in the dosing regimen of the interferon lambda and/or lonafarnib components during the course of treatment. In some embodiments, the subject receives a dose reduction of interferon lambda and/or lonafarnib, in that one or more later doses is a lower dose than one or more earlier doses. In some embodiments, a dose is reduced if the subject exhibits unacceptable side effects. In some embodiments, a subject may receive multiple dose reductions during the course of treatment with interferon lambda, lonafarnib, and ritonavir. In some embodiments, the dosage administered to the subject is not reduced before 8 weeks of treatment at the first dosage (e.g., at a first dosage of 180 mcg QW), or before 1 week, 2 weeks, 3 weeks, 4 weeks, 5 weeks, 6 weeks, or 7 weeks of treatment at the first dosage. In some embodiments, the dosage administered to the subject is not reduced before 9-12 weeks of treatment at the first dosage (e.g., at a first dosage of 180 mcg QW). The lonafarnib dose may be reduced at the same time (or times) as the interferon lambda dose, and/or the lonafarnib dose may be reduced at different times from the interferon lambda dose. In some embodiments, the dose of interferon lambda is reduced, and the dose of the lonafarnib is not reduced. In some embodiments, the dose of lonafarnib is reduced, and the dose of interferon lambda is not reduced.

In some embodiments, the interferon lambda, lonafarnib, and ritonavir therapy comprises administering to the subject interferon lambda at a first interferon lambda dose (e.g., 180 micrograms per week) and lonafarnib at a first lonafarnib dose (e.g., 50 mg BID) for a first treatment period, followed by administering to the subject interferon lambda at a second interferon lambda dose (e.g., 120 micrograms per week) and lonafarnib at a second lonafarnib dose (e.g., 25 mg BID) for a second treatment period. In some embodiments, the length of time for the first treatment period is the same as the length of time for the second treatment period. In some embodiments, the ritonavir dose remains the same in both treatment periods.

In some embodiments, the first treatment period and the second treatment period are different lengths of time. In some embodiments, the first treatment period is longer than the second treatment period. In some embodiments, the second treatment period is longer than the first treatment period.

Subjects may receive interferon lambda, lonafarnib, and ritonavir therapy for a predetermined time, until not tolerated, or until an endpoint is reached. Treatment may be continued on a continuous daily basis for at least two to three months. In some embodiments, the interferon lambda, lonafarnib, and ritonavir therapy is continued for at least 30 days, e.g., at least 60 days, at least 90 days, at least 120 days, at least 150 days, or at least 180 days. In some embodiments, the interferon lambda, lonafarnib, and ritonavir treatment is continued for at least 6 months, e.g., at least 7 months, at least 8 months, at least 9 months, at least 10 months, at least 11 months, at least one year, at least 15 months, at least 18 months, or at least 2 years. In some embodiments, the therapy is continued for at least 6 weeks, e.g., at least 12 weeks, at least 18 weeks, at least 24 weeks, at least 30 weeks, at least 36 weeks, at least 42 weeks, at least 48 weeks, at least 60 weeks, at least 72 weeks, at least 84 weeks, or at least 96 weeks. In other embodiments, the interferon lambda, lonafarnib, and ritonavir therapy is continued for the remainder of the subject's life or until administration is no longer effective in maintaining the virus at a sufficiently low level to provide meaningful therapeutic benefit.

In accordance with the methods herein, some HDV subjects will respond to interferon lambda, lonafarnib and ritonavir therapy, as described herein, by clearing virus to undetectable levels. In some embodiments, for subjects in which HDV RNA levels are below the level of detection, treatment is suspended unless and until the HDV levels return to detectable levels. Other subjects will experience a reduction in viral load and improvement of symptoms but will not clear the virus to undetectable levels but will remain on therapy for a defined period of time (e.g., for about 1 year, about 2 years, about 3 years, or longer) or so long as the therapy provides therapeutic benefit.

In some embodiments, treatment with interferon lambda, lonafarnib, and ritonavir therapy results in a reduction of HDV viral load in the subject of at least 1.5 lop) HDV RNA copies/mL serum when measured after 24 weeks of treatment. In some embodiments, treatment with interferon lambda, lonafarnib, and ritonavir therapy results in a reduction of HDV viral load in the subject of at least 2.0 log₁₀ HDV RNA copies/mL serum when measured after 24 or 48 weeks of treatment. In some embodiments, treatment with interferon lambda, lonafarnib, and ritonavir therapy results in a reduction of HDV viral load in the subject of at least 2.5 log₁₀ HDV RNA copies/mL serum when measured after 24 weeks of treatment.

In some embodiments, treatment with interferon lambda, lonafarnib, and ritonavir therapy results in a sustained reduction of HDV viral load (e.g., a decrease of at least 1.5 log₁₀ HDV RNA IU/mL serum, at least 2.0 log₁₀ HDV RNA copies/mL serum, or at least 2.5 log₁₀ HDV RNA IU/mL serum, or a decrease in HDV RNA to undetectable levels) that is sustained for a period of time (e.g., 1 month, 3 months, 6 months, 1 year or longer) while the course of treatment is still ongoing. In some embodiments, treatment with interferon lambda, lonafarnib, and ritonavir therapy results in a sustained reduction of HDV viral load that is sustained for a period of time (e.g., 1 month, 3 months, 6 months, 1 year or longer) after the course of treatment is finished. In some embodiments, the interferon lambda, lonafarnib, and ritonavir therapy results in HDV RNA levels (e.g., serum HDV RNA levels or plasma HDV RNA levels) below 1,000 copies/mL. In some embodiments, the HDV RNA levels remain below 1,000 copies/mL for at least one month, e.g., at least three months, at least one year, or longer. In some embodiments, the course of treatment results in HDV RNA levels (e.g., serum HDV RNA levels or plasma HDV RNA levels) below 100 copies/mL. In some embodiments, the HDV RNA levels remain below 100 copies/mL for at least one month, at least three months, at least one year, or longer. The phrase “remains below” refers to remaining below an initial measured value (e.g., 100 copies/mL or 100 IU/mL) for a period of time, for example, at 1 month (or another specified time) a viral load measurement taken at least 1 month (or at the other specified time) after determination of the initial measured value is no higher than the initial value. In some embodiments, the subject does not receive interferon lambda therapy during the specified time. In some embodiments, the subject does not receive any anti-HDV treatment during the specified time.

In some embodiments, therapy as disclosed herein is continued for a period of time until HDV RNA levels are below 3 log₁₀ HDV RNA copies/mL (below 1,000 copies/mL), or sometimes until HDV RNA levels are below 2 log₁₀ HDV RNA copies/mL (below 100 copies/mL) or below the level of detection. In some embodiments, therapy is continued for a period of time (such as 1 to 3 months or longer) after viral load has dropped to acceptably low levels (e.g., undetectable levels). In some embodiments, therapy is continued until the HDV viral load is reduced to undetectable levels.

In some embodiments, a subject treated according to the methods described herein exhibits a reduction in HDV viral load to undetectable levels during the course of treatment, and the subject maintains the reduction in HDV viral load to undetectable levels for at least 12 weeks after the end of treatment. In some embodiments, a subject treated according to the methods described herein exhibits a reduction in HDV viral load to undetectable levels during the course of treatment, and the subject maintains the reduction in HDV viral load to undetectable levels for at least 24 weeks after the end of treatment.

In some embodiments, the subject's HDV titer rises from baseline prior to dropping below baseline during the course of treatment. In some embodiments, the subject's HDV level rises to more than 150% of baseline, or more than 200% of baseline. In some embodiments, the rise in the titer is from 25-50% of baseline, from 25-100% of baseline, or from 50-200% of baseline. In some embodiments, the rise in the titer occurs within 2 weeks after initiation of therapy. In some embodiments, the subject's elevated HDV titer drops to below baseline within 2 weeks, or within 3 weeks, of initiation of therapy.

In some embodiments, a subject treated according to the methods described herein exhibits an improvement in one or more liver function parameters. In some embodiments, the improved liver function is an improvement in one or more serum markers (e.g., one, two, three, four, five, six or more markers), such as serum albumin, bilirubin, alanine aminotransferase (ALT), aspartate aminotransferase (AST), prothrombin, alfa2-macroglobulin, apolipoproteinAl, haptoglobin, gamma-glutamyl transpeptidase (GGT). In some embodiments, a subject treated according to the methods described herein exhibits an improvement in liver fibrosis (e.g., as assessed by biopsy with histological analysis, transient ultrasound elastography (e.g., FibroScan®), or magnetic resonance elastography). In some embodiments, treatment results in an improvement of at least 5%, e.g., at least 10%, at least 20%, at least 30%, at least 40%, at least 50%, at least 70%, at least 75%, at least 80%, or at least 100%, or between 5-50%, between 10-80%, or between 50-100% improvement in one or more liver function parameters (e.g., an improvement in serum marker(s) or an improvement in liver fibrosis) in the subject as compared to prior to the initiation of treatment. In some embodiments, treatment results in an improvement in one or more liver function parameters (e.g., an improvement in serum marker(s) or an improvement in liver fibrosis) to the level of a healthy control subject that is not infected with HDV or HBV. In some embodiments, the subject exhibits an improvement in serum ALT levels to a level that is within the upper limit of normal.

In some embodiments, a subject treated according to the methods described herein exhibits a reduction in HBV viral load compared to the baseline level at the initiation of treatment and/or compared to a similarly infected subject not receiving treatment effective to reduce the subject's HDV viral load. In some embodiments, treatment results in a reduction of at least 1 log₁₀ in HBV viral load.

Prior to treatment, the subject's HDV and/or HBV viral load is measured to determine the baseline viral load. After a period of treatment (e.g., after 12 weeks of treatment), the subject's viral load is reduced compared to baseline. In some embodiments, after a period of treatment (e.g., after 12 weeks of treatment), the subject's viral load is substantially reduced compared to baseline, such as to very low levels or to an undetectable level. In some embodiments, treatment results in an at least 2 lop) reduction of HBV viral load. In some embodiments, subjects treated according to the methods described herein exhibit a reduction in HBsAg levels or an improvement in clearance of HBsAg antigen. Prior to treatment the subject's HBsAg level is measured to determine a baseline. After a period of treatment (e.g., after 12 weeks of treatment), the subject's HBsAg level is reduced compared to baseline. In some embodiments, subjects treated according to the methods described herein exhibits the presence of anti-HBs antibody.

Subjects receiving interferon lambda, lonafarnib, and ritonavir therapy according to the present disclosure may also be treated with one or more other antiviral agents such as nucleoside and nucleotide analogs, compounds used to treat HBV infections, and other agents.

In some embodiments, a subject who is administered interferon lambda, lonafarnib, and ritonavir therapy is treated with an antiviral agent that is used for the treatment of HBV. Anti-HBV medications that are currently approved, with the exception of interferons, inhibit reverse transcriptase and are nucleoside or nucleotide analogs. These medications, while effective against HBV DNA, are not effective against HDV as they do not clear HBsAg, which HDV needs to replicate. Currently approved anti-HBV nucleoside/nucleotide analogs include lamivudine (Epivir-HBV®, Zeffix®, or Heptodin®), adefovir dipivoxil (Hepsera®), entecavir (Baraclude®), telbivudine (Tyzeka® or Sebivo®), clevudine (Korea/Asia), tenofovir (Viread® or Vemlidy®). In some embodiments, a subject who is administered interferon lambda therapy is also administered a nucleoside or nucleotide analogs, such as but not limited to lamuvidine, adefovir, telbivudine, entecavir, tenofovir, or clevudine. In some embodiments, the subject is receiving nucleoside or nucleotide analog therapy prior to the onset of interferon lambda therapy. In some embodiments, nucleoside or nucleotide analog therapy is initiated at the start of interferon lambda therapy or during the course of interferon lambda therapy.

The following embodiments are contemplated. As used below, any reference to a series of embodiments is to be understood as a reference to each of those embodiments disjunctively (e.g., “Embodiments 1-4” is to be understood as “Embodiments 1, 2, 3, or 4”).

Embodiment 1 is a method of treating a hepatitis delta virus (HDV) infection in a subject, the method comprising a drug regimen comprising subcutaneously administering to the subject a therapeutically effective amount of interferon lambda once per week and administering to the subject a therapeutically effective amount of lonafarnib and ritonavir daily, wherein the interferon lambda, the lonafarnib, and the ritonavir are administered until one or more of:
a sustained reduction of HDV viral load is reached,
a decrease in HDV RNA to undetectable levels is reached,
the interferon lambda, the lonfarnib, and the ritonavir have been administered for at least 12 weeks, or
the interferon lambda, the lonfarnib, and the ritonavir have been administered for at least 24 weeks, or
the interferon lambda, the lonafarnib, and the ritonavir have been administered for at least 48 weeks.

Embodiment 2 is a method of treating a hepatitis delta virus (HDV) in a subject, the method comprising:
administering to the subject a therapeutically effective amount of each of interferon lambda, lonafarnib, and ritonavir for a first treatment period, wherein the interferon lambda is administered at a first interferon lambda dosage, the lonafarnib is administered at a first lonafarnib dosage, and the ritonavir is administered at a first ritonavir dosage; and
administering to the subject a therapeutically effective amount of each of lonafarnib and ritonavir for a second treatment period after the first treatment period, wherein the lonafarnib is administered at a second lonafarnib dosage and the ritonavir is administered at a second ritonavir dosage.

Embodiment 3 is the embodiment of any of embodiment(s) 1-2, wherein the interferon lambda comprises pegylated interferon lambda.

Embodiment 4 is the embodiment of any of embodiment(s) 1-3, wherein the interferon lambda comprises interferon lambda-1a.

Embodiment 5 is the method of any of embodiment(s) 1-4, wherein the interferon lambda, the lonafarnib, and the ritonavir are administered for at least 12 weeks, at least 24 weeks, at least 36 weeks, at least 48 weeks, at least 54 weeks, or between 12 weeks and 96 weeks.

Embodiment 6 is the method of any of embodiment(s) 1-5, wherein the interferon lambda is administered at a dose of 180 micrograms once a week, 90 micrograms twice per week, 80 micrograms twice per week, or 180 micrograms per week.

Embodiment 7 is the method of any of embodiment(s) 1-6, wherein the interferon lambda is administered at a dose of 120 micrograms once a week, 60 micrograms twice per week, 70 micrograms twice per week, or 120 micrograms per week.

Embodiment 8 is the method of any of embodiment(s) 1-7, wherein there is a decrease of HDV RNA by >2 log from baseline at 24 weeks after initiation of administration.

Embodiment 9 is the method of any of embodiment(s) 1-8, wherein a subject has undetectable HDV RNA in serum at week 12 and at week 24 of a post-treatment follow-up.

Embodiment 10 is the method of any of embodiment(s) 1-9, wherein the subject has a reduction in histologic inflammatory scores (modified HAI) by at least two points at end of treatment with no progression in histologic fibrosis.

Embodiment 11 is the method of any of embodiment(s) 1-10, wherein the subject has normalization of serum ALT at one or more of: the end of treatment, week 12 of a post-treatment follow-up, and week 24 of the post-treatment follow-up.

Embodiment 12 is the method of embodiment 11, wherein the subject is female and exhibits an ALT level <20 IU/L, or wherein the subject is male and exhibits an ALT level is <31 IU/L.

Embodiment 13 is the method of any of embodiment(s) 1-12, wherein the subject has reduction in serum ALT by >50% of baseline at one or both of: week 12 of a post-treatment follow up, or week 24 of the post-treatment follow up.

Embodiment 14 is the method of any of embodiment(s) 1-13, wherein the subject has reduction in hepatic venous pressure gradient (HVPG) measurements by >25% of baseline or normalization of HVPG (<5 mm Hg) at the end of treatment.

Embodiment 15 is the method of any of embodiment(s) 1-14, wherein the subject has reduction in Fibroscan® transient elastography values by >25% of baseline at the end of treatment.

Embodiment 16 is the method of any of embodiment(s) 1-15, wherein the subject has a reduction in fibrosis score to F1 at the end of treatment.

Embodiment 17 is the method of any of embodiment(s) 1-16, wherein the subject has a reduction in fibrosis score at the end of treatment of at least 1 level, 2 levels, 3 levels, or 4 levels.

Embodiment 18 is the method of any of embodiment(s) 1-17, wherein the subject has loss of HBsAg from serum at one or more of: the end of treatment, week 12 of a post-treatment follow-up, or at week 24 of the post-treatment follow-up.

Embodiment 19 is the method of any of embodiment(s) 1-18, wherein the subject has changes in quantitative HBsAg levels compared to baseline at one or both of: the end of treatment and at week 24 of a post treatment follow-up.

Embodiment 20 is the method of any of embodiment(s) 1-19, wherein after 12 weeks of treatment at least about 81% of subjects have a median HDV RNA decline from baseline of at least 3.6 log IU/mL.

Embodiment 21 is the method of any of embodiment(s) 1-20, wherein after 12 weeks of treatment a majority of subjects have a median RNA decline of between about 2.6-4.2 log IU/mL.

Embodiment 22 is the method of any of embodiment(s) 1-21, wherein after 12 weeks of treatment at least about 24% of subjects have undetectable HDV RNA.

Embodiment 23 is the method of any of embodiment(s) 1-22, wherein after 12 weeks of treatment at least about 24% of subjects have HDV RNA below the lower limit of quantification (BLOQ).

Embodiment 24 is the method of any of embodiment(s) 1-23, wherein at the end of treatment at least about 73% of subjects have HDV RNA decline of at least about 3.4 log IU/mL.

Embodiment 25 is the method of any of embodiment(s) 1-24, wherein at the end of treatment a majority of subjects have HDV RNA decline of from about 2.9-about 4.5 log IU/mL

Embodiment 26 is the method of any of embodiment(s) 1-25, wherein at the end of treatment 37-42% of subjects achieve undetectable HDV RNA.

Embodiment 27 is the method of any of embodiment(s) 1-26, wherein at the end of treatment at least about 11-16% of subjects achieve BLOQ.

Embodiment 28 is the method of any of embodiment(s) 1-27, wherein at the end of treatment at least about 95-96% of subjects achieved >2 log decline of HDV RNA during 24 weeks of treatment.

Embodiment 29 is the method of any of embodiment(s) 1-28, wherein >50% of subjects achieve undetectable or BLOQ HDV RNA levels.

Embodiment 30 is the method of any of embodiment(s) 1-29, wherein the method further comprises administering to the subject a nucleoside analog or nucleotide analog.

Embodiment 31 is the method of embodiment 30, wherein the nucleoside analog or nucleotide analog is lamuvidine, adefovir, telbivudine, entecavir, or tenofovir.

Embodiment 32 is the method of any of embodiment(s) 1-31, wherein the subject has compensated liver disease with or without cirrhosis.

Embodiment 33 is the method of embodiment 32, wherein the subject has compensated liver disease with cirrhosis.

Embodiment 34 is the method of any of embodiment(s) 1-33, wherein the interferon lambda is administered at a dose of 180-120 mcg per week, the lonafarnib is administered at 50 mg BID, and the ritonavir is administered at 200 mg QD.

Embodiment 35 is the method of any of embodiment(s) 1-34, wherein the subject has at least an 81% probability of having a median HDV RNA decline from baseline of at least 3.6 log IU/mL after 12 weeks of treatment.

Embodiment 36 is the method of any of embodiment(s) 1-35, wherein the subject is likely to have a median HDV RNA decline of between about 2.6-4.2 log IU/mL after 12 weeks of treatment.

Embodiment 37 is the method of any of embodiment(s) 1-36, wherein the subject has at least a 24% probability of having undetectable HDV RNA after 12 weeks of treatment.

Embodiment 38 is the method of any of embodiment(s) 1-37, wherein the subject has at least a 24% probability of having HDV RNA below the lower limit of quantification (BLOQ) after 12 weeks of treatment.

Embodiment 39 is the method of any of embodiment(s) 1-38, wherein the subject has at least a 73% probability of having HDV RNA decline of at least about 3.4 log IU/mL by the end of treatment.

Embodiment 40 is the method of any of embodiment(s) 1-39, wherein the subject is likely to have HDV RNA decline of from about 2.9-about 4.5 log IU/mL by the end of treatment.

Embodiment 41 is the method of any of embodiment(s) 1-40, wherein the subject has at least a 37% probability of having undetectable HDV RNA by the end of treatment.

Embodiment 42 is the method of any of embodiment(s) 1-41, wherein the subject has at least about a 16% probability of having BLOQ by the end of treatment.

Embodiment 43 is the method of any of embodiment(s) 1-42, wherein, at the end of treatment, the subject has at least about a 95% probability of having >2 log decline of HDV RNA during 24 weeks of treatment.

Embodiment 44 is the method of any of embodiment(s) 1-43, wherein the subject has a >50% probability of achieving undetectable or BLOQ HDV RNA levels by the end of treatment.

Embodiment 45 is the method of any of embodiment(s) 1-44, wherein about 11.5% of subjects dose reduced one or more of the pegylated interferon lambda-1a or the lonafarnib.

Embodiment 46 is the method of any of embodiment(s) 1-45, wherein about 15.4% of subjects discontinued the administration of pegylated interferon lambda-1a.

Embodiment 47 is the method of any of embodiment(s) 1-46, wherein the amount of interferon lambda or lonafarnib administered to the subject is reduced before the end of treatment.

Embodiment 48 is the method of any of embodiment(s) 1-47, wherein the subject has at least about an 11.5% probability of requiring a reduction of the amount of interferon lambda or lonafarnib administered to the subject.

Embodiment 49 is the method of any of embodiment(s) 1-48, wherein the administration of interferon lambda is discontinued before the end of treatment.

Embodiment 50 is the method of any of embodiment(s) 1-49, wherein the subject at least about a 15.4% probability of requiring discontinuation of the administration of interferon lambda.

Embodiment 51 is the method of any of embodiment(s) 1-50, wherein the subject has HDV RNA below the lower limit of quantification (BLOQ) after 12 weeks of treatment.

Embodiment 52 is the method of any of embodiment(s) 1-51, wherein the subject has an HDV RNA decline of at least 3.4 log IU/mL at the end of treatment.

Embodiment 53 is the method of any of embodiment(s) 1-52, wherein the subject has an HDV RNA decline of from about 2.9-about 4.5 log IU/mL at the end of treatment

Embodiment 54 is the method of any of embodiment(s) 1-53, wherein the subject has undetectable HDV RNA at the end of treatment.

Embodiment 55 is the method of any of embodiment(s) 1-54, wherein the subject has HDV RNA BLOQ at the end of treatment.

Embodiment 56 is the method of any of embodiment(s) 1-55, wherein the subject has >2 log decline of HDV RNA during 24 weeks of treatment at the end of treatment.

Embodiment 57 is the method of any of embodiment(s) 1-56, wherein the subject has HDV RNA BLOQ.

Embodiment 58 is the method of any of embodiment(s) 1-57, wherein the first lonafarnib dosage is equal to the second lonafarnib dosage.

Embodiment 59 is the method of any of embodiment(s) 1-58, wherein the first lonafarnib dosage is less than the second lonafarnib dosage.

Embodiment 60 is the method of any of embodiment(s) 1-59, wherein the first lonafarnib dosage is greater than second lonafarnib dosage.

Embodiment 61 is the method of any of embodiment(s) 1-60, wherein the first ritonavir dosage is equal to the second ritonavir dosage.

Embodiment 62 is the method of any of embodiment(s) 1-61, wherein the first ritonavir dosage is less than the second ritonavir dosage.

Embodiment 63 is the method of any of embodiment(s) 1-62, wherein the first ritonavir dosage is greater than second ritonavir dosage.

Embodiment 64 is the method of any of embodiment(s) 1-63, wherein the first treatment period is equal to the second treatment period.

Embodiment 65 is the method of any of embodiment(s) 1-64, wherein the first treatment period is less than the second treatment period.

Embodiment 66 is the method of any of embodiment(s) 1-65, wherein the first treatment period is greater than the second treatment period.

Embodiment 67 is a method of assessing impact of a therapeutic agent targeting hepatitis beta virus (HBV) on an hepatitis delta virus (HDV) infection in a subject having a co-infection of HBV and HDV, the method comprising: administering the therapeutic agent to the subject for a treatment period; measuring an amount of HBsAg in a biological sample from the subject after the treatment period; and determining if the amount of HBsAg in the biological sample is undetectable; wherein if the amount of HBsAg is undetectable, the therapeutic agent impacts the HDV infection.

Embodiment 68 is the method of embodiment 67, wherein the method further comprises: determining a baseline amount HDV RNA in the subject; measuring an amount of HDV RNA in a biological sample from the subject after the treatment period; and determining if the amount of HDV RNA in the biological sample is reduced relative to the baseline amount of HDV RNA.

### EXAMPLES

### Example 1

Provided herein is an open-label clinical trial treating 26 adult patients with chronic delta hepatitis with peginterferon lambda-1a (LMD) combined with the prenylation inhibitor lonafarnib (LNF) boosted with ritonavir (RTV) (generally, “LMD/LNF/RTV”).for 24 weeks. This study is referred to as the Lambda Interferon Combination Therapy (LIFT) study. Patients have undergone baseline testing including blood draws, imaging, safety consults, and liver biopsy with portal pressure measurements prior to starting therapy. During therapy, all patients have been monitored for quantitative HDV RNA and HBV DNA levels as well as routine safety measures and liver function tests. After 24 weeks, therapy was stopped and patients again underwent repeated testing including blood draws, imaging, and safety consults. After stopping therapy, patients are being followed for an additional 24 weeks.

Subjects are 18 years or older and have the presence of anti-HDV in serum; presence of quantifiable HDV RNA in serum at three pre-treatment time points with a mean HDV RNA level >2 log₁₀ above the lower limit of quantification (LLOQ) of the HDV RNA assay and the demonstration of chronicity as evidenced by the presence of HDV RNA in serum for 6 months, or presence of anti-HDV antibody 6 months.

Subjects having decompensated liver disease, defined by bilirubin >4 mg/dL, albumin <3.0 gm/dL, prothrombin time >2 sec prolonged, or history of bleeding esophageal varices, ascites or hepatic encephalopathy, were not enrolled. Subjects with ALT levels greater than 1000 U/L (>25 times ULN) were not enrolled. Patients with an absolute neutrophil count <1000/dL and platelets <75,000/dL were excluded from the study. Subjects were also not enrolled if they had significant systemic or major illnesses other than liver disease, including, but not limited to, congestive heart failure, renal failure (eGFR<50 ml/min), organ transplantation, serious psychiatric disease or depression, or active coronary artery disease; systemic immunosuppressive therapy within the previous 2 months before enrollment; evidence of another form of liver disease in addition to viral hepatitis (for example autoimmune liver disease, primary biliary cirrhosis, primary sclerosing cholangitis, Wilson disease, alcoholic liver disease, ongoing drug induced liver disease, nonalcoholic steatohepatitis (but not steatosis), hemochromatosis, or alpha-1-antitrypsin deficiency); active substance abuse, such as alcohol, inhaled or injection drugs within the previous year; evidence of hepatocellular carcinoma. Subjects were not enrolled if they have elevated AFP; evidence of concurrent hepatitis C infection with positive serum HCV RNA; any experimental therapy or pegylated interferon therapy within 6 months prior to enrollment; active, serious autoimmune disease such as systemic lupus erythematosus, ulcerative colitis, Crohn's disease or rheumatoid arthritis.

To prevent the possibility of a hepatitis B flare during the study, all patients were treated with nucleos(t)ide analogues while on the study (24 weeks of entecavir or tenofovir+lonafarnib/ritonavir/lambda and 24 weeks of entecavir or tenofovir during the post-therapy monitoring phase). Patients who were not on nucleos(t)ide analogues prior to starting lonafarnib/ritonavir/lambda were started on either entecavir or tenofovir (which are first-line nucleos(t)ide analogues recommended by the AASLD and EASL for the treatment of HBV) prior to instituting HDV therapy (to reduce the risk of a hepatitis B flare). HBV viral loads were monitored after starting nucleoside analogue therapy at each outpatient visit during the entire course of study participation. Adequate HBV suppression (serum HBV DNA level <2000 IU/mL) on nucleoside analogue therapy was required prior to starting HDV therapy to allow for improved determination of response to experimental HDV therapy.

All patients were initiated on lonafarnib 50 mg orally twice daily, ritonavir 100 mg orally twice daily, and interferon lambda 180 mcg subcutaneously weekly. Patients were initiated on therapy, for example, one or two days after the liver biopsy and remained in the clinical center for 72 hours after induction of therapy for observation of side effects, administration of medication, and timed blood draws to facilitate analysis of virological response kinetics and pharmacokinetic analysis. During the admission, frequent blood sampling were performed (0, 6, 12, 18, 24, 36, 48 and 72 hours after the first dose) for viral kinetics, pharmacokinetics, and storage.

Under certain conditions, subjects were dose reduced as follows: Interferon lambda dose reduced from 180 mcg to 120 mcg, and lonafarnib dose reduced from 50 mg to 25 mg. Doses were reduced, for example, if a subject's experienced an adverse event that is greater than or equal to a Grade 3 that is one or more of: related to the lambda or lonafarnib, possibly related to the lambda or lonafarnib, possibly related to the lambda or lonafarnib, and not clinically significant. Doses were reduced, for example, if a subject experienced depression, had new ocular symptoms, hematologic abnormalities or creatinine clearance of less than 50 mL/min.

In this ongoing study, 26 adult patients with chronic HDV and quantifiable HDV RNA in serum (lower limit of quantitation <40 IU/mL) have completed treatment with LMD/LNF/RTV for 24 weeks and are undergoing per-protocol post-therapy monitoring for 24 weeks. Patients were 62% male, median age of 40 years and included Asian (54%), White (31%) and African (15%) subjects. Median baseline evaluations included: ALT (62 IU/mL), AST (47 IU/mL), Ishak Fibrosis (3), modified HAI inflammation (9), HBV DNA (<21 IU/mL) and log HDV RNA (4.74 IU/mL). After 12 weeks of therapy, the median HDV RNA decline from baseline was 3.4 log IU/mL (IQR: 2.9-3.8, p<0.0001) with 7 patients (27%) achieving undetectable HDV RNA and 6 patients (23%) with HDV RNA below the lower limit of quantification (BLOQ). At the end of therapy at 24 weeks, the median HDV RNA decline was 3.4 log IU/mL (IQR: 2.9-4.5, p<0.0001) with 11 patients (42%) achieving undetectable HDV RNA and 3 patients (11%) BLOQ. 25 of 26 patients (96%) achieved >2 log decline during 24 weeks of therapy. Adverse events were mostly mild to moderate and included GI related side effects, weight loss, hyperbilirubinemia, and anemia. Therapy was dose reduced in 3 patients and discontinued in 4 patients.

Triple combination therapy with peginterferon lambda-1a (LMD) combined with lonafarnib (LNF) boosted with ritonavir (RTV) in chronic HDV patients appears to be safe and tolerable for up to 6 months in most patients. After 24 weeks, almost all patients achieve >2 log decline in HDV RNA during therapy with >50% achieving undetectable or BLOQ HDV RNA levels.

### Example 2

Administration of peginterferon lambda (Lambda) for 48 weeks was shown to induce a durable virologic response (HDV RNA below limit of quantification at 24 weeks post-treatment) in 36% of patients with HDV and compensated liver disease (Phase 2 LIMT Study, NCT02765802). See also International Application No. PCT/US2019/048038 for description of this therapy. Impact of Lambda therapy on liver histology was not assessed in the LIMT study. In this example, two case reports of regression of liver fibrosis following treatment in HDV patients from the LIMT study are provided.

Two patients from the LIMT study, who had liver biopsies prior to study randomization, treated with subcutaneous injections of Lambda 180 mcg once weekly for 48 weeks, followed by a 24 week follow-up period, were evaluated with liver biopsy 18 months after last Lambda injection. Liver biopsies, staged according to the ISHAK scoring system, were compared with results of historical biopsies of the patients. HDV RNA viral load and ALT levels were assessed at baseline (BL), end of treatment (EOT) and end of study (EOS). Fibroscan® was performed at BL and EOS.

FibroScan is a specialized ultrasound machine that measures fibrosis (scarring) and steatosis (fatty change) in the liver. The Fibroscan device (Echosens) works by measuring shear wave velocity. In this technique, a 50-MHz wave is passed into the liver from a small transducer on the end of an ultrasound probe. The probe also has a transducer on the end that can measure the velocity of the shear wave (in meters per second) as this wave passes through the liver. The shear wave velocity can then be converted into liver stiffness, which is expressed in kilopascals. Essentially, the technology measures the velocity of the sound wave passing through the liver and then converts that measurement into a liver stiffness measurement; the entire process is often referred to as liver ultrasonographic elastography. See additional discussion in Afdhal, N.H., Gastroenterol Hepatol (N Y). 8(9): 605-607 (2012).

In Patient 1, a 64-year-old male, HDV RNA level was 3.7 log₁₀ at BL, became undetectable at EOT and rebounded to 2.6 log₁₀ at EOS. ALT was 169 U/L at BL, declined to 55 U/L at EOT and remained at 54 U/L at EOS. Fibroscan® scores declined from 20 kPa at BL to 9.9 kPa at EOS. Comparison of historical liver biopsy to biopsy following Lambda treatment was significant for reduction in fibrosis score from F5 (incomplete cirrhosis) to F1 (mild portal fibrosis).

In Patient 2, a 37-year-old female, HDV RNA was 4.9 log₁₀ at BL, became undetectable at EOT and rebounded to 3.6 log₁₀ at EOS. ALT was 159 U/L at BL, declined to 44 U/L at EOT and peaked to 162 U/L at EOS. Fibroscan® score of 7.7 kPa at BL increased to 11.1 kPa at EOS. Comparison between the patient's historical and post-treatment biopsies showed reduction in fibrosis score from F4 (marked bridging fibrosis) to F1.

This is the first report demonstrating fibrosis regression following finite duration therapy with Lambda in patients with chronic HDV, the most severe form of hepatitis for which there is no approved treatment. These case studies suggest clinical benefit in the liver after 48 weeks of Lambda therapy in the absence of HDV RNA clearance.

### Example 3

As new agents are in development for HBV, we sought to predict their effect on HDV co-infection. While many agents appear to have various effects on the level of serum HBV DNA, the majority have significantly less effect on HBsAg levels. HBsAg (also known as the Australia antigen) is the surface antigen of the hepatitis B virus (HBV). It indicates current hepatitis B infection. Even for those agents that offer the prospect of more than one log reduction in HBsAg, it is unclear if this would have a meaningful impact on a co-existing HDV infection, as the only requirement from HBV is a source of HBsAg, however small. HDV appears to be more efficient than HBV in co-opting the HBsAg that is present in a co-infected cell.

To further address the predicted effect of new agents in development for HBV on HDV, this study examined HDV RNA levels as a function of HBsAg levels for progressively lower thresholds of HBsAg in a cohort from a single site of HDV-infected patients in Ulaanbaatar, Mongolia.

Paired quantitative HBsAg (qHBsAg) [limit of detection (LOD)=0.03 IU/mL] and HDV RNA (LOD=50 IU/mL) results in 57 patients with elevated ALT from the database of The Liver Center in Ulaanbaatar, Mongolia were available for analysis. Patients were grouped by the following qHBsAg levels: <50 IU/mL, between 50 and 100 IU/mL, and between 100 and 1000 IU/mL. Mean HDV RNA and mean ALT values were then determined for each qHBsAg threshold. The results of this analysis are summarized in Table 1 below.

Even with very low quantitative HBsAg levels (i.e. <100 IU/mL), HDV RNA persists with elevated ALT levels, consistent with continued liver damage. HBV therapies in development that are unable to completely eradicate HBsAg are unlikely to have a significant effect on a co-existing HDV infection.

TABLE 1

| | | qHBsAg (IU/mL) | | |
|---|---|---|---|---|
| | All | <50 | 50-100 | 100-1000 |
| # of Patients | 57 | 4 | 4 | 49 |
| Female | 16 | 0 | 1 | 15 |
| Male | 41 | 4 | 3 | 34 |
| Mean qHBsAg | 427.9 | 16.3 | 68.8 | 490.8 |
| (range, IU/mL) | (0.52-942) | (0.52-35) | (58-76) | (145-942) |
| Mean Log HDV | 3.99 | 2.66 | 2.79 | 4.19 |
| RNA | (0.90-6.78) | (1.31-4.07) | (1.00-3.99) | (0.90-6.78) |
| (range, IU/mL) | | | | |
| ALT | 113 | 83 | 66 | 120 |
| (range, U/L) | (26-734) | (36-197) | (40-123) | (26-734) |

All publications and patents cited in this specification are herein incorporated by reference as if each individual publication or patent were specifically and individually indicated to be incorporated by reference and are incorporated herein by reference to disclose and describe the methods and/or materials in connection with which the publications are cited.

It should be understood that although the present invention has been specifically disclosed by certain aspects, embodiments, and optional features, modification, improvement and variation of such aspects, embodiments, and optional features can be resorted to by those skilled in the art, and that such modifications, improvements and variations are considered to be within the scope of this disclosure.

The inventions have been described broadly and generically herein. Each of the narrower species and subgeneric groupings falling within the generic disclosure also form part of the invention. In addition, where features or aspects of the invention are described in terms of Markush groups, those skilled in the art will recognize that the invention is also thereby described in terms of any individual member or subgroup of members of the Markush group.

1. A method of treating a hepatitis delta virus (HDV) infection in a subject, the method comprising subcutaneously administering to the subject a therapeutically effective amount of interferon lambda once per week and administering to the subject a therapeutically effect amount of lonafarnib and ritonavir daily, wherein the interferon lambda, the lonafarnib, and the ritonavir are administered until one or more of:
a sustained reduction of HDV viral load is reached,
a decrease in HDV RNA to undetectable levels is reached,
the interferon lambda, the lonafarnib, and the ritonavir have been administered for at least 12 weeks,
the interferon lambda, the lonafarnib, and the ritonavir have been administered for at least 24 weeks, or
the interferon lambda, the lonafarnib, and the ritonavir have been administered for at least 48 weeks.

2. The method of claim 1, wherein the interferon lambda comprises pegylated interferon lambda.

3. The method of claim 1, wherein the interferon lambda comprises interferon lambda-1a.

4. The method of claim 1, wherein the interferon lambda, the lonafarnib, and the ritonavir are administered for at least 12 weeks, at least 24 weeks, at least 36 weeks, at least 48 weeks, at least 54 weeks, or from 12 weeks to 96 weeks.

5. The method of claim 1, wherein the interferon lambda is administered at a dose of 180 micrograms once per week, 90 micrograms twice per week, 80 micrograms twice per week, or 180 micrograms per week.

6. The method of claim 1, wherein the interferon lambda is administered at a dose of 120 micrograms per week, 60 micrograms twice per week, 70 micrograms twice per week, or 120 micrograms per week.

7. The method of claim 1, wherein the subject has a decrease of HDV RNA in serum of >2 log from baseline at 24 weeks after initiation of administration.

8. The method of claim 1, wherein the subject has undetectable HDV RNA in serum at week 12 and at week 24 of a post-treatment follow-up.

9. The method of claim 1, wherein the subject has a reduction in histologic inflammatory scores (modified HAI) by at least two points at end of treatment with no progression in histologic fibrosis.

10. The method of claim 1, wherein the subject has normalization of serum ALT at one or more of: the end of treatment, week 12 of a post-treatment follow-up, and week 24 of the post-treatment follow-up.

11. The method of claim 8, wherein the subject is female and exhibits an ALT level <20 IU/L, or wherein the subject is male and exhibits an ALT level <31 IU/L.

12. The method of claim 1, wherein the subject has reduction in serum ALT by >50% of baseline at one or both of: week 12 of a post-treatment follow up or week 24 of the post-treatment follow up.

13. The method of claim 1, wherein the subject has reduction in hepatic venous pressure gradient (HVPG) measurements by >25% of baseline or normalization of HVPG (<5 mm Hg) at the end of treatment.

14. The method of claim 1, wherein the subject has reduction in Fibroscan® transient elastography values by >25% of baseline at the end of treatment.

15. The method of claim 1, wherein the subject has a reduction in fibrosis score to F1 at the end of treatment.

16. The method of claim 1, wherein the subject has a reduction in fibrosis score at the end of treatment of at least 1 level, 2 levels, 3 levels, or 4 levels.

17. The method of claim 1, wherein the subject has loss of HBsAg from serum at one or more of: the end of treatment, week 12 of a post-treatment follow-up, or week 24 of the post-treatment follow-up.

18. The method of claim 1, wherein the subject has changes in quantitative HBsAg levels compared to baseline at one or both of: the end of treatment and at week 24 of a post treatment follow-up.

19. (canceled)

20. (canceled)

21. (canceled)

22. (canceled)

23. (canceled)

24. (canceled)

25. (canceled)

26. (canceled)

27. (canceled)

28. (canceled)

29. The method of claim 1, wherein the method further comprises administering to the subject a nucleoside analog or nucleotide analog.

30. The method of claim 29, wherein the nucleoside analog or nucleotide analog is lamuvidine, adefovir, telbivudine, entecavir, or tenofovir.

31. The method of claim 1, wherein the subject has compensated liver disease with or without cirrhosis.

32. The method of claim 31, wherein the subject has compensated liver disease with cirrhosis.

33. The method of claim 1, wherein the interferon lambda is administered at a dose of 180-120 mcg per week, the lonafarnib is administered at 50 mg BID, and the ritonavir is administered at 200 mg QD.

34. (canceled)

35. (canceled)

36. A method of treating a hepatitis delta virus (HDV) in a subject, the method comprising:
administering to the subject a therapeutically effective amount of each of interferon lambda, lonafarnib, and ritonavir for a first treatment period, wherein the interferon lambda is administered at a first interferon lambda dosage, the lonafarnib is administered at a first lonafarnib dosage, and the ritonavir is administered at a first ritonavir dosage; and
administering to the subject a therapeutically effective amount of each of lonafarnib and ritonavir for a second treatment period after the first treatment period, wherein the lonafarnib is administered at a second lonafarnib dosage and the ritonavir is administered at a second ritonavir dosage.

37. A method of assessing impact of a therapeutic agent targeting hepatitis beta virus (HBV) on an hepatitis delta virus (HDV) infection in a subject having a co-infection of HBV and HDV, the method comprising:
administering the therapeutic agent to the subject for a treatment period;
measuring an amount of HBsAg in a biological sample from the subject after the treatment period; and
determining if the amount of HBsAg in the biological sample is undetectable;
wherein if the amount of HBsAg is undetectable, the therapeutic agent impacts the HDV infection.

38. The method of claim 37, wherein the method further comprises:
determining a baseline amount HDV RNA in the subject;
measuring an amount of HDV RNA in a biological sample from the subject after the treatment period; and
determining if the amount of HDV RNA in the biological sample is reduced relative to the baseline amount of HDV RNA.
